(19) United States
(12) Patent Application Publication
(10) Pub. No.: US 2023/0310190 A1
(43) Pub. Date: Oct. 5, 2023

(54) HAND WASHING ASSISTIVE DEVICE

(71) Applicant: SIMPLE ASSIST THERAPEUTICS, LLC, Rehoboth, DE (US)

(72) Inventor: Rebecca Della-Rodolfa, Rehoboth Beach, DE (US)

(21) Appl. No.: 18/205,563

(22) Filed: Jun. 4, 2023

(51) Int. Cl. A61F 4/00; A46B 5/00; A46B 15/00

(52) U.S. Cl. CPC A61F 4/00; A46B 5/0095; A46B 5/0041; A46B 15/0055; A46B 5/005; A46B 2200/1013

(57) ABSTRACT

A hand washing assistive device includes a prosthetic hand, a supportive arm, a support mechanism, and a mechanism for removably coupling the support member to an underlying surface. The supportive arm is coupled to the prosthetic hand. The support member is coupled to the supportive arm and has a connector that permits the supportive arm to be movable. The prosthetic hand is configured to provide a complementary hand to a user's hand for washing purposes.

### CROSS-REFERENCE TO RELATED APPLICATION

This application claims priority to U.S. Provisional Patent Application No. 63/327,349, filed on Apr. 4, 2022, the disclosure of which is incorporated herein by reference in its entirety.

### FIELD

The present invention concerns an assistive device to aid a disabled individual to wash their hand.

### BACKGROUND

Living with an upper limb difference creates a unique set of challenges during the most routine of tasks that able-bodied people take for granted. For example, persons with limb differences often find it hard to wash their functioning hand. It is desired to assist those with a permanent disability or temporary inability relating to an upper extremity, to achieve an effective handwashing. Persons who are missing a limb, such as a hand, or an arm and hand, have difficulty washing their remaining hand because they do not have an opposing hand to wash against. Those who have suffered the loss of use of a limb, such as through the inability to use a limb because of stroke, injury, or otherwise, have similar difficulties.

### BRIEF DESCRIPTION OF THE DRAWINGS

FIG. 1 depicts a perspective view of a first embodiment of an assistive device according to the invention with the assistive device being attached to a countertop in a retracted position next to a sink;

FIG. 2 depicts a perspective view of the assistive device of FIG. 1 in a position of use over a sink;

FIG. 3 depicts a side view of the assistive device of FIG. 1;

FIG. 4 depicts a top view of the assistive device of FIG. 1; and

FIG. 5 depicts a top plan view of another embodiment of an assistive device according to the invention.

### SUMMARY

An assistive device for assisting in handwashing for a user having a deficiency in one arm or hand is disclosed. The assistive device is positioned adjacent a sink and a user uses the assistive device to clean their hand.

### DETAILED DESCRIPTION

The present invention is directed toward assisting a person having only one working hand, even if that hand is affected by pain or weakness. It is useful for those missing a hand as well as those who suffer from the inability to use a limb due to a disability, such as a stroke, injury, or surgery.

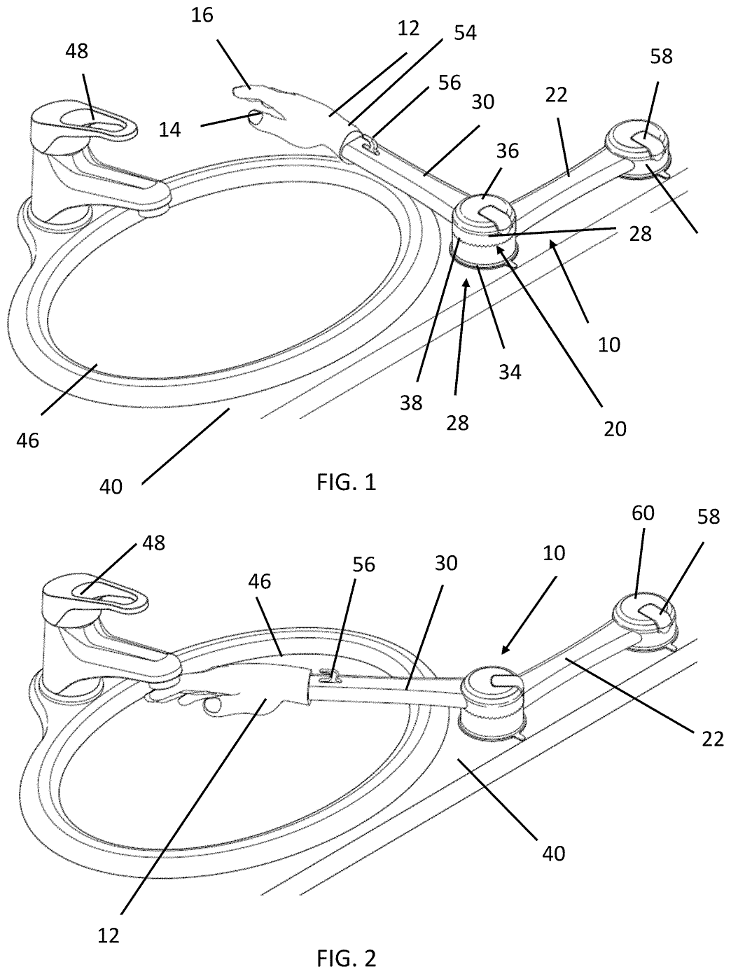

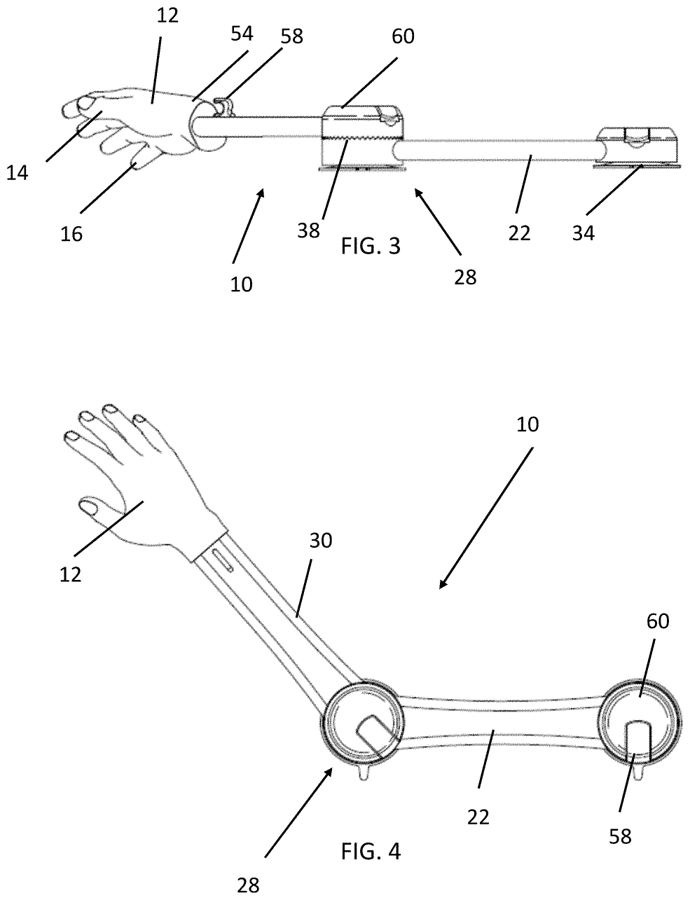

As shown in FIGS. 1-4, the assistive device 10 is made in two parts. The first part is in the form of a hand 12. The hand 12 includes a thumb 14 and fingers 16 that are slightly spread apart, and the hand 12 may be angled A, such as at a 30-degree or 45-degree angle to make it easier for the user to use the device 10. The hand 12 may be made of a soft, flexible plastic, rubber, or foam so that it has some volume and flexibility to feel as natural as possible. The hand 12 may include a support structure (not shown) that is surrounded by a foam or other material to make the hand 12 soft to the touch. The support structure may be fixed or may be bendable into various positions. For example, the support structure may permit the fingers 16 to be separated or the thumb 14 to be moved, or the hand 12 to be bent.

The fingers 16 are flexible and they permit a user to push their fingers between the fingers 16 of the assistive device 10 to provide an effective cleaning of the user's hand. The hand 12 may come in different types of materials or may include covers that provide different types of materials, as described below.

The second part 20 of the assistive device 10 includes a base 22 and a supportive arm 30. The base 22 of device 10, as shown in the figures, includes a first longitudinally extending, rod or bar-like member 22 that is connected at a joint 28 to an arm 30. The arm 30 is connected at the free end 32 to the hand 12 of the first part. The first bar-like member 22 includes a first suction cup 34 at one end and a second suction cup 34 at the opposite end. The arm 30 is coupled to the first bar-like member 22 at the second suction cup 34 via a rotatable member 36. The second suction cup 34 includes the rotatable member 36 with locking teeth 38 that permits the arm 30 to rotate toward and away from the first bar-like member 22. The use of two suction cups 34 along with a rotatable arm member 36 permits the assistive device 10 to be firmly attached to a countertop 40 so that a user can easily use the hand 12 to wash the user's hand and so that the hand 12 accepts pressure applied by the user's hand.

The suction cups 34 may be heavy duty suction cups for attaching to the countertop 40. The first bar-like member 22 can be the same length as the arm 30 or could be shorter or longer.

At the end 32 of arm 30, a connector (not shown) is provided for connecting to the hand 12. Any type of connector can be used. For example, arm 30 may include a post and the hand 12 may include a receptacle (not shown) that slides over the post. It is preferred that the hand 12 may be locked in place so that it is firmly attached, which will aid the user in being able to apply pressure to the hand 12 during washing to get effective cleaning of the user's hand.

Alternatively, the hand 12 could be held in place without a locking mechanism. In one embodiment, the locking mechanism for locking the hand 12 to the arm 30 requires the user to slide the hand 12 into the arm 30 and to turn the hand 12 until it locks in place. To remove the hand 12, a slight turn is used to unlock it, and it can then be pulled from the arm 30. When the hand 12 is locked in place, the thumb 14 will be positioned in a slightly upward angle.

As shown in FIGS. 1 and 2, the hand 12 is designed to swing over the opening of a sink 46 adjacent a faucet 48. The hand 12 may be swung into the sink 46 for use and swung away from the sink 46 so that it is positioned outside the sink when not in use. The suction cup 34 that is coupled to the arm 30 has a rotatable mechanism that permits the arm 30 and hand 12 to be rotated towards and away from the sink 46. The hand 12 is not attached to the countertop 40 other than via the arm 30.

The mechanism that couples the arm 30 to the bar-like member 22 may be a gear mechanism that includes teeth 38 that permits the arm 30 to be held in position. The gear mechanism may include teeth 38 on a top portion that mate with teeth 38 on a bottom portion thereof. The gear mechanism can permit the arm to swing up to 180 degrees, for example. The user may pull up, for example, on the arm 30 to release its locked position to rotate it away from or towards the sink 46. The arm 30 may simply be rotated with a small amount of force to move it in and out of the sink 46 without having to lift it up. Other types of mechanisms can be used for locking the arm 30 in position over the sink 46.

In use, the hand 12 should be positioned over a sink 46 and be preferably near the faucet 48 so that a user can apply water and soap to the user's hand and to the assistive device 10. By hanging the hand 12 of the device 10 over the sink 46, water and soap suds will be contained in the sink 46 during the handwashing process.

The hand 12 is connected to the arm 30 so that someone with one functioning hand can move the arm 30 in and out of position. This is true even if the user's functioning hand is weak or is painful.

As discussed above, the hand 12 may include a variety of covers (not shown), which may fit like gloves over the hand 12. The covers may include a gentle cover, a standard cover, and a heavy-duty cover, for example.

A gentle cover may be made of a microfiber material and may be used by those who have fragile skin and/or are hyper-sensitive. Hyper-sensitivity may be caused by skin breakdown, neuropathy, or other issues. The gentle cover may be used daily for simple hand washing by all users.

The standard cover may be made of a spongy material that is reasonably coarse for improved scrubbing ability. The standard cover is for use by a user who requires a more abrasive cleaning surface than the gentle cover but does not need an overly abrasive surface. This type of cover may be used by a user who may cook, or who gets butter or cooking oil on the user's hand, or who has handled raw meat, chicken, or fish, for example.

The heavy-duty cover may be made of a tougher material and provides ultra-scrubbing ability to the user. This cover may be used by a user who is not concerned with skin breakdown and needs a heavy-duty washing of the user's hand. This may be a person who gets grease or motor oil on their hand from working on a car, or someone who works in the yard or garden and gets dirt under their nails, or sap on their hands. Other covers and materials may be used, as desired.

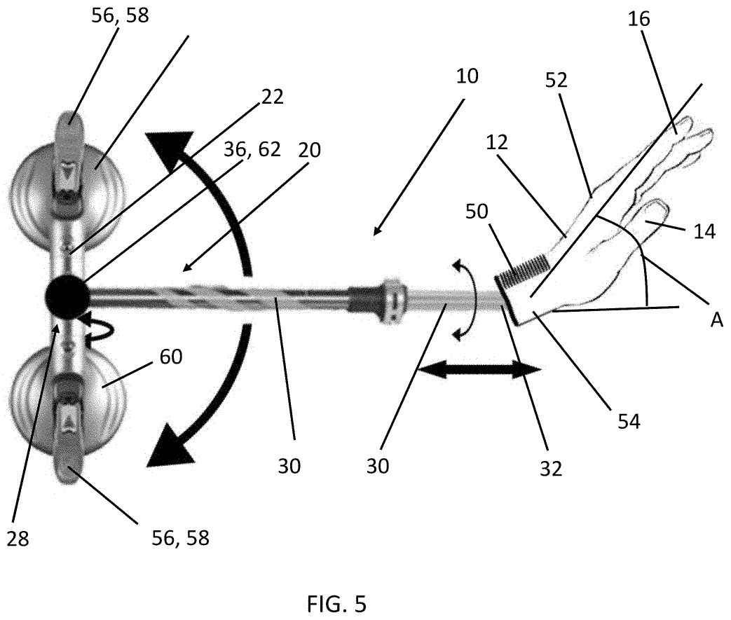

The hand 12 may be provided with small brush bristles 50 that can be used as a nail brush to clean under the user's fingernails. The brush bristles can be attached directly to the hand 12 or could, alternatively, be coupled to a cover. One possible location for the brush bristles would be along the knuckles 52 of the hand 12 or cover. Another possible location for the bristles could be at the wrist 54 of the hand 12, as shown in FIG. 5. The nail brush can be made removable and may be attached to the device 10 in any known manner, such as by using snaps or other fasteners that permit the nail brush 50 to be removed, reapplied, and/or replaced when it has worn out.

The device 10, when assembled, is sturdy enough to allow an effective hand-wash, even if a heavy scrubbing of the user's hand is needed. Each of the covers may be dishwasher or washing machine safe.

If covers are not utilized, abrasive properties may be built directly into the material of the hand 12. For example, if the hand 12 is molded, abrasive materials may be molded into the hand 12 on the surface of the hand 12 or at different locations on the hand 12. The entire hand 12 may have the same level of abrasiveness or parts of the hand 12 could have different levels of abrasiveness, as desired.

It is preferred that the suction cups 34 of the device 10 are not released from the countertop 40 too frequently to ensure longevity of the device 10, which is one reason why the hand 12 is rotatable into and out of position in a sink 46. The hand 12 may be rotatable into various positions, other than just a 30 or 45-degree angle. This may permit the hand 12 to be used to hold things, for example, among other uses.

There may also be a small hook 56 built into or attached on the top of the arm 30. The hook 56 is shown in FIGS. 1-4 near the hand 12 and positioned on an upper surface of the arm 30. The hook 12 could alternatively or in addition thereto be positioned on the bar-like member 22 of the device 10, if desired. Alternatively, one of the connectors that are used to couple the parts of the device 10 together could include hooks 56 that the user can use to pull up their sleeve, as shown in FIG. 5. It is intended that the hook 56 be used to pull up a sleeve of a user prior to handwashing. The positioning of the hook 56 will allow for a comfortable movement of the user's arm to move their sleeve up their arm.

The suction cups 34 are preferably strong enough so that a user of the sleeve hook 56 does not accidentally unlock the suction cups 34 from the underlying surface 40. The nail brush 50 and sleeve hook 56 may be labeled with wording or images (not shown) to instruct the user concerning the purpose of the tools.

Referring now to FIG. 5, an alternative embodiment of the assistive device is shown. While this embodiment is different in structure from the embodiment discussed in connection with FIGS. 1-4, it has very similar properties. Moreover, features from each embodiment may be equally incorporated into the other embodiment.

In the assistive device 10 of FIG. 9, the second part 20, which includes the suction cup base or housing 60 and the supportive arm 30 includes at least two lock to grip suction cups 34 that are connected to a straight member 22 that is positioned between the two suction cups 34. The suction cups 34 may have diameters such as between 3.5 inches and 4.75 inches, for example. Other sized suction cups 34 may alternatively be used. The suction cups 34 may be spaced with an overall length of approximately 11 inches to 15 inches apart, for example. Other lengths may alternatively be used. The length may include the lockdown levers 58, which may protrude past the edges of the suction cups 34. The suction cups 34 and their associated housings 60 could be injection molded nylon (or similar plastic). Alternatively, the suction cups and their housings could be cast aluminum.

The straight member 22 may be a single piece that is part of the rigid housings 60 of the suction cups 34 or could be a separate tube or rod that attaches to the housings 60. The straight member 22 may locked in place once a desired location is found. The straight member 22 may be locked in place using snap buttons (spring clips), like those used for telescoping tubing. Alternatively, other types of connectors could be used to fix the length of the straight member 22.

The straight member 22 includes a hub 62 on the straight member 22 that may provide rotation in two different axes. The hub 62 may also provide a means for locking the straight member 22 in place with a quick release lever or other types of mechanism, such as friction hinges and the like.

The straight member 22 may include a separate tube or arm 30, such as an aluminum tube, that the hand 12 is mounted to at a free end of the tube 32. The hand 12 may be mounted with snap buttons, quick release, or other mechanical fasteners that may permit easy actuation of the hand 12. The angle of the hand 12 relative to the tube 30 may be adjustable. Alternatively, the angle of the hand 12 relative to the tube may be fixed.

The aluminum tube 30 may telescope from inside a slightly larger diameter aluminum tube or directly through a quick release locking feature on the straight member's rotating hub 62. If using the two telescoping tubes method, then the larger tube would simply attach to the straight member's rotating hub with a snap button, quick release or other mechanical fastener that could be easily actuated with one hand.

The hand 12 may be sized so that both small and large hands could use it without difficulty. Possible dimensions for use by both small and large hands are approximately 7.75 inches long and 3.75 inches wide. Other dimensions may alternatively be used. The hand 12 may include a rigid or semi-rigid structure that is covered by a softer material like silicone rubber or TPE. The rubber coating could have various textures applied to its surface to improve its scrubbing properties. The fingers 16 of the hand 12 may be firm but with decent flexion so users' fingers can fit between the fingers 16 of the hand for cleaning of the user's hand.

The nail brush for cleaning under fingernails could be positioned near the wrist of the hand 12 or on the aluminum tube 30, close to where the hand 12 is attached to the tube 30. Other locations for the nail brush could also be provided.

The embodiment shown in FIG. 5 includes a hook 56 in the form of a knob that is used to couple the suction cup housing 60 to the countertop or to connect the straight member 30 between the suction cups 34. This hook or knob 56 would be used to snag the cuff of a long-sleeved shirt to pull the sleeve up and out of the way before hand washing.

To set up and use the device:
1. The user positions the suction cup housings 60 with a central straight connecting member having a rotating hub 62 attached thereto on a flat surface, such as a countertop. The device 10 is preferably positioned as close as possible to the left or right of a sink, depending on which hand the user will be cleaning. The suction cups 34 are locked down using the suction cup locking levers 58.
2. The user may then attach the telescoping tube to the rotating hub 62. Then the hand 12 is attached to the telescoping tube, if necessary.
3. The user may then position the hand 12 in the direction of the faucet, extend and rotate the hand 12 into a comfortable position and lock the hand 12 in place with, for example, quick release lever(s). Since most kitchen faucets can rotate back and forth, the hand may be best positioned off to one side to permit the user to soap up, scrub, and then rotate the faucet over the hand for a final rinse.

The device 10 may be sold with one hand attachment 12 in the material of the buyer's choice and then additional covers may be purchased. If covers are not utilized, additional hands 12 having different textures or abrasiveness levels may be purchased and coupled to the arm 30. Alternatively, different abrasiveness levels may be positioned at different locations on the hand 12, if desired, so that a user moves the user's hand to the desired abrasiveness level on the hand for effective cleaning.

While not shown, the assistive device 10 may alternatively be provided with a single suction cup 34 and a single arm 30 that is connected directly to the hand 12.

The assistive device 10 may also be used for other uses, such as for holding a dish or other item in the sink so that a user can wash the dish with the user's hand. The hand 12 could be removed to provide a platform for placing a dish to be washed, for example. The assistive device may be positioned on either side of the sink, with the location selected based upon which hand is to be washed.

The hand 12 may come in different sizes depending upon hand size and shape. A small handed person can likely use a larger hand 12, but a larger handed person may have difficulty using a smaller hand 12. Thus, multiple sizes of hands may be sold separately. Alternatively, an average sized hand may be determined and sold as a universally shaped hand 12 so that users with both small and large hands can use the same device 10.

Fingers 16 on the hand 12 must be firm but have some flexion so that a user's fingers can fit between the fingers 16 of the hand 12 for cleaning purposes. The hand 12 may be reversible so that it can be positioned on either side of the sink 46 so that it is usable by either a left hand or a right hand of a user.

An abrasive surface on the hand 12 can be achieved in numerous ways, such as through over molding. In the process of over molding, the hand shape is molded using a support substrate or interior structure that provides some rigidity to the hand 12. The substrate is placed in the mold and an elastomer is molded over the substrate to form the skin of the hand 12 that a user would contact with the user's hand. The elastomeric skin may be any desired thickness and may also have abrasive properties to assist the user with hand washing.

As discussed above, another way to provide different textures or levels of abrasiveness is to apply a glove (not shown) over the molded hand 12. The glove would be removable, washable, and replaceable. The glove could be washable, for example, in a dishwasher. When the glove wears out, it can be replaced. Different types of fabrics can be used to provide different levels of abrasiveness. For example, an ultra-soft glove could be used for cleaning sensitive or fragile skin and could be labeled a “Gentle” version of the fabric, a fabric with light texture for a light scrub could be labeled a “Standard” fabric, and a fabric with a rough texture could provide heavy scrubbing capability. This latter fabric could be labeled an “Ultra Scrub” version of the fabric.

According to one embodiment of the invention, a hand washing assistive device includes a prosthetic hand, a supportive arm, a support mechanism, and a mechanism for removably coupling the support member to an underlying surface. The supportive arm is coupled to the prosthetic hand. The support member is coupled to the supportive arm and has a connector that permits the supportive arm to be movable. The prosthetic hand is configured to provide a complementary hand to a user's hand for washing purposes.

The assistive device may include a coupling mechanism positioned between the support member and the supportive arm that permits the prosthetic hand to be movable and to be locked into a desired position. The mechanism for removably coupling the support member to a surface may be one or more suction cups. The one or more suction cups may be coupled to a housing and the support member may be is coupled to the housing. The one or more suction cups may include a lock down mechanism for locking the suction cups to an underlying surface.

The prosthetic hand may be one or more of soft, pliable, and abrasive. The prosthetic hand may comprise a support structure and a skin structure positioned over the support structure. The skin structure may be rubber or soft plastic and may be molded over the support structure. Alternatively, a separate glove-like member may be positioned over the support structure. The skin structure may include an abrasive or textured surface to aid in cleaning of a user's hand.

The assistive device may also include a nail brush coupled to the assistive device that permits a user to clean their nails. The nail brush may be removable and replaceable. The prosthetic hand may include fingers having knuckles and a wrist. The nail brush may be coupled to one or both of the knuckles and wrist. The assistive device may also include a sleeve hook coupled to the assistive device for assisting a user in pushing up their sleeve.

The prosthetic hand may have a wrist with a receptacle extending longitudinally into the wrist of the prosthetic hand. The support arm may include a post for coupling with the receptacle of the prosthetic hand to join the wrist to the hand in a semi-permanent, but removable position.

The prosthetic hand may be angled at an angle relative to a longitudinal axis of the supportive arm. The angle of the prosthetic hand relative to the longitudinal axis of the supportive arm may be adjustable. The support arm may be telescopic so that its length may be lengthened and shortened. Alternatively, the length of the support member may be adjustable so that it may be lengthened and shortened. The supportive arm may be movable along the length of the support member and rotatable about the support member.

A method for washing a hand using the assistive device may include coupling the assistive device to a surface adjacent a sink, positioning the prosthetic hand over the sink near a faucet of the sink, and applying soap and water to a user's hand and contacting the prosthetic hand to provide an opposing surface for washing a user's hand. The positioning step may include adjusting an angle of the hand relative to the supportive arm so that the hand is in a proper position for use against another hand during hand washing.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. The articles “a,” “an,” and “the,” should be interpreted to mean “one or more” unless the context indicates the contrary.

What is claimed is:

1. A hand washing assistive device comprising:
a prosthetic hand;
a supportive arm coupled to the prosthetic hand;
a support member coupled to the supportive arm and having a connector that permits the supportive arm to be movable; and
a mechanism for removably coupling the support member to an underlying surface, wherein the prosthetic hand is configured to provide a complementary hand to a user's hand for washing purposes.

2. The assistive device of claim 1, further comprising a coupling mechanism positioned between the support member and the supportive arm that permits the prosthetic hand to be movable and to be locked into a desired position

3. The assistive device of claim 1, wherein the mechanism for removably coupling the support member to a surface is one or more suction cups.

4. The assistive device of claim 3, wherein the one or more suction cups are coupled to a housing and the support member is coupled to the housing.

5. The assistive device of claim 3, wherein the one or more suction cups include a lock down mechanism for locking the suction cups to an underlying surface.

6. The assistive device of claim 1, wherein the prosthetic hand is one or more of soft, pliable, and abrasive.

7. The assistive device of claim 1, wherein the prosthetic hand comprises a support structure and a skin structure positioned over the support structure.

8. The assistive device of claim 7, wherein the skin structure is rubber or soft plastic and is molded over the support structure, or the skin structure is glove-like and is positioned over the support structure.

9. The assistive device of claim 7, wherein the skin structure includes an abrasive or textured surface to aid in cleaning of a user's hand.

10. The assistive device of claim 1, further comprising a nail brush coupled to the assistive device that permits a user to clean their nails.

11. The assistive device of claim 10, wherein the nail brush is removable and replaceable.

12. The assistive device of claim 1, wherein the prosthetic hand includes fingers having knuckles and a wrist and the nail brush is coupled to one or both of the knuckles and wrist.

13. The assistive device of claim 1, further comprising a sleeve hook coupled to the assistive device for assisting a user in pushing up their sleeve.

14. The assistive device of claim 1, wherein the prosthetic hand has a wrist with a receptacle extending longitudinally into the wrist of the prosthetic hand, and the support arm includes a post for coupling with the receptacle of the prosthetic hand to join the wrist to the hand in a semi-permanent, but removable position.

15. The assistive device of claim 1, wherein the prosthetic hand is angled at an angle relative to a longitudinal axis of the supportive arm.

16. The assistive device of claim 15, wherein the angle of the prosthetic hand relative to the longitudinal axis of the supportive arm is adjustable.

17. The assistive device of claim 1, wherein the support arm is telescopic so that its length may be lengthened and shortened, or the length of the support member is adjustable so that it may be lengthened and shortened.

18. The assistive device of claim 1, wherein the supportive arm is movable along the length of the support member and rotatable about the support member.

19. A method for washing a hand using the assistive device of claim 1 comprising:
coupling the assistive device to a surface adjacent a sink;
positioning the prosthetic hand over the sink near a faucet of the sink;
applying soap and water to a user's hand and contacting the prosthetic hand to provide an opposing surface for washing a user's hand.

20. The method of claim 19, wherein the positioning step includes adjusting an angle of the hand relative to the supportive arm so that the hand is in a proper position for use against another hand during hand washing.
